(19) United States
(12) Patent Application Publication
(10) Pub. No.: US 2024/0238050 A1
(43) Pub. Date: Jul. 18, 2024

(54) BAYONET REGISTRATION TOOL/PROBE

(71) Applicant: DIGITAL SURGERY SYSTEMS, INC., Goleta, CA (US)

(72) Inventors: Alan Fridman, Goleta, CA (US); Alan Siu, Ventura, CA (US); George C. Polchin, Santa Barbara, CA (US); Stephen C. Minne, Santa Barbara, CA (US); Saurabh Kotian, Goleta, CA (US); Thomas Kanusky, Santa Barbara, CA (US)

(21) Appl. No.: 18/563,213

(22) PCT Filed: May 20, 2022
(86) PCT No.: PCT/US2022/030271

§ 371 Date: Nov. 21, 2023

Related U.S. Application Data
(60) Provisional application No. 63/191,561, filed on May 21, 2021.

(51) Int. Cl. A61B 34/20; A61B 17/00; A61B 90/00

(52) U.S. Cl. CPC A61B 34/20; A61B 17/00; A61B 90/361; A61B 2017/00199; A61B 2017/00725; A61B 2034/2055; A61B 2034/2068; A61B 2090/3618; A61B 2090/363; A61B 2090/3983

(57) ABSTRACT

The present disclosure provides new and innovative systems and apparatuses for a surgical registration probe that provides improved detection by a surgical navigation system (e.g., localizer). An example apparatus includes a surgical registration probe; a localizer configured to track the surgical registration probe; and a surgical visualization system (e.g., localizer) configured to display a surgical site responsive to the tracking. The surgical registration probe may include: a marker shaft with at least one marker or fiducial; wherein the marker or fiducial causes the localizer to detect the surgical registration probe; and a probe shaft having at least three sections that are bent at a defined angle with respect to each other. In some embodiments, the apparatus may further comprise a calibration plate for securing the surgical registration probe.

### RELATED APPLICATION

The present application claims priority to and the benefit of U.S. Provisional Patent Application 63/191,561, filed May 21, 2021, the entirety of which is incorporated herein by reference.

### BACKGROUND

Many surgeries use live video data and/or virtual templates or preoperative images to assist a surgeon. Some known surgical systems provide surgical navigation in which the live video data, virtual templates, or preoperative images are aligned with an orientation of a patient as viewed by the surgeon. Surgical navigation requires that live patient are registered to a reference frame coordinate space. FIG. 1 is a diagram of a known straight registration probe comprising a shaft 102 connected to a frame 104 containing infrared (IR) sensitive spherical markers. As shown in FIG. 1, the frame includes at least three fiducials 106 or markers that are detectable by an optical tracking system (localizer) and a computer. A navigation system is able to visualize and identify the patient using a reference frame, rigidly attached to a Mayfield clamp that holds a patient's head, and a localizer (e.g., an IR camera connected to the navigation system).

Known navigation systems align the live patient information to, for example, DICOM image data such as MRI/CT scans acquired pre-surgery. This enables surgeons to navigate a patient anatomy using DICOM data. The straight reference probe is commonly used for registering patients in a supine, lateral, or sitting position. However, using straight reference probes is difficult or not possible for patients in a prone position. FIG. 2 shows diagrams of a patient in a prone position. In this position, a surgeon has to tilt a registration probe downward to capture points on a patient's face. To achieve good registration accuracy, it is recommended to collect points from facial structures (e.g., easily identifiable rigid landmarks) of the patient instead of the scalp or back of the patient.

However, the localizer is typically not able to easily detect a registration probe that is pointed in a downward orientation. Oftentimes, the registration probe is located below a patient plane, meaning that the localizer has to have a direct line of sight to the registration probe. Further, surgical staff or surgical equipment often block this limited line of sight and it is not reasonable to maintain this line of sight free of obstacles during surgery. This can be especially challenging since the localizer camera is generally located far and above the patients plane in order to capture a maximum volume for navigation during surgery.

The localizer can be moved down below a patient's plane to visualize the registration tool. However, the reference frame which is usually located above the patients head might go out of the localizer's line of sight in this case and registration might fail. Moreover, it is often difficult for a surgeon to crouch down or bend over during surgery to align a registration probe with a prone patient's face during surgery, which is ergonomically challenging. It can also cause user frustration if registration fails or results in low accuracy forcing the surgeon to redo the registration steps and ultimately not use a surgical navigation/visualization system at all.

### SUMMARY

The present disclosure provides new and innovative systems and apparatuses for a surgical registration probe that provides improved detection by a surgical navigation system (e.g., localizer). In an example, a surgical registration probe is disclosed that includes: a marker shaft with at least one marker or fiducial; and a probe shaft having at least three sections that are bent at a defined angle with respect to each other. For example, the marker shaft may include four markers or fiducials. A first segment may be connected to the probe shaft (e.g., at an angle between 20 and 120 degrees), a second segment may be connected to the first segment (e.g., at an angle between 20 and 120 degrees), and a third segment may be connected to the second segment (e.g., at an angle between 20 and 120 degrees). In some embodiments, the surgical registration probe may include a mirror connected to at least one section of the probe shaft and positioned to reflect an end of the probe shaft. In yet another embodiment, an end of the probe shaft may include a spherical ball for placement against a patient's skin. Furthermore, the surgical registration probe can be reusable after sterilization (e.g., autoclave, ethylene oxide (EtO), hydrogen peroxide (H2O2), etc.).

In another example, a calibration plate is disclosed for securing a surgical registration probe. The calibration plate may include: a rectangular plate comprising a top surface, a bottom surface, and a hole at the center of the rectangular plate having an opening through the top surface and an opening through the bottom surface. The top surface may display one or more fiducials (e.g., detectable markers) that may cause or allow a surgical navigation system (e.g., a localizer) to track the calibration plate (e.g., during surgery). The hole may be configured to secure a tip of the surgical registration probe via the opening through the bottom surface. The surgical registration probe may comprise a bayonet surgical registration probe comprising a marker shaft portion, a bent shaft portion, and the tip. In some aspects, the marker shaft portion may comprise one or more additional fiducials causing the localizer to track the surgical registration probe. The calibration plate may be configured to secure the bayonet surgical registration probe by causing the marker shaft portion to be perpendicular to the rectangular plate, and causing the bent shaft portion to be underneath the bottom surface of the rectangular plate. The opening through the top surface may be smaller than the tip of the surgical registration probe. Furthermore, the opening through the bottom surface may be larger than the tip of the surgical registration probe. For example, the hole may be configured as a countersink to properly hold the tip of the surgical registration probe. In some embodiments, the calibration plate may further include a plurality of stilts protruding from the bottom surface of the rectangular plate. The plurality of stilts may elevate the rectangular plate. The elevation may be configured to allow a curved shaft of the surgical registration probe to be placed beneath the bottom surface of the rectangular plate. In a further embodiment, the calibration plate may include a plurality of slits extending inwards from the sides of the rectangular plate. The plurality of slits may be configured to hold a shaft of the surgical registration probe

In yet another example, an apparatus is disclosed for improved localizer visibility to a surgical registration probe. The apparatus includes a surgical registration probe (e.g., a bayonet surgical registration probe); a localizer configured to track the surgical registration probe; and a surgical visualization system (e.g., localizer) configured to display a surgical site responsive to the tracking. The surgical registration probe may include: a marker shaft with at least one marker or fiducial; wherein the marker or fiducial causes the localizer to detect the surgical registration probe; and a probe shaft having at least three sections that are bent at a defined angle with respect to each other. In some embodiments, the apparatus may further comprise a calibration plate for securing the surgical registration probe.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE FIGURES

FIG. 1 is an illustration of a known registration probe.

FIG. 2 is a diagram of a patient in a prone position.

FIG. 3 is a diagram of an example bayonet surgical registration tool, according to an example embodiment of the present disclosure.

FIG. 4 is another diagram of an example bayonet surgical registration tool, according to an example embodiment of the present disclosure.

FIG. 5 is a further diagram of an example bayonet surgical registration tool, according to an example embodiment of the present disclosure.

FIG. 6 is a diagram of the example bayonet surgical registration tool in a user's hand, according to an example embodiment of the present disclosure.

FIGS. 7 to 9 are diagrams of different possible dimensions and angles for an example bayonet surgical registration tool, according to an example embodiment of the present disclosure.

FIGS. 10 to 12 are diagrams of different proposed designs for an example bayonet surgical registration tool, according to an example embodiment of the present disclosure.

FIGS. 13 to 16 are diagrams of an example bayonet surgical registration tool with a mirror, according to an example embodiment of the present disclosure.

FIG. 17 is a diagram of an example calibration plate to secure the bayonet surgical registration tool, according to an example embodiment of the present disclosure.

FIG. 18 is a diagram of an example slitted calibration plate to more effectively secure the surgical registration tool, according to an example embodiment of the present disclosure.

FIG. 19 shows diagrams of example add-on patient markers configured to mate the bayonet surgical registration tool, according to an example embodiment of the present disclosure.

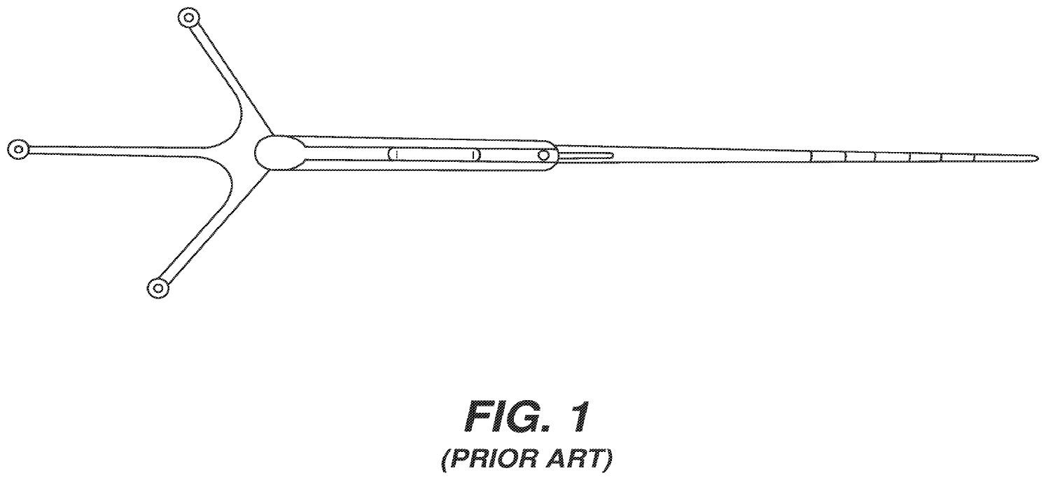

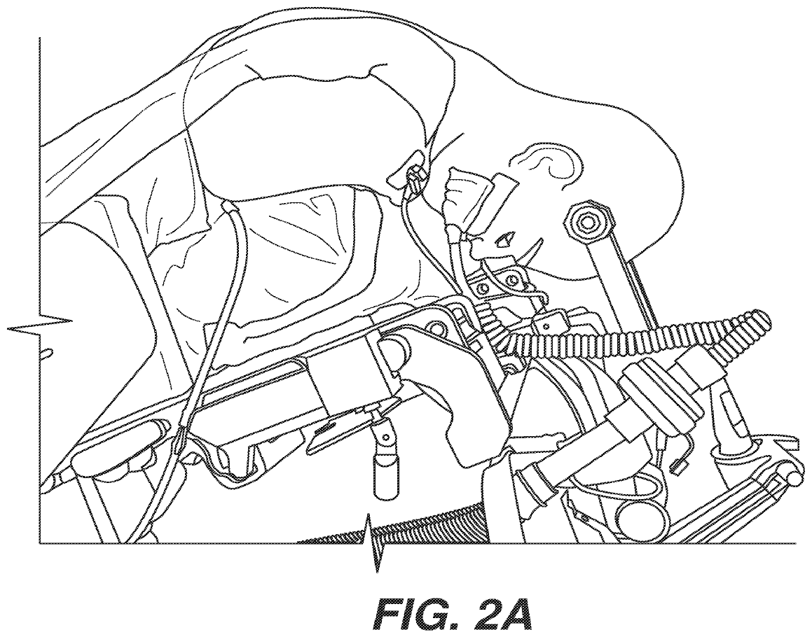

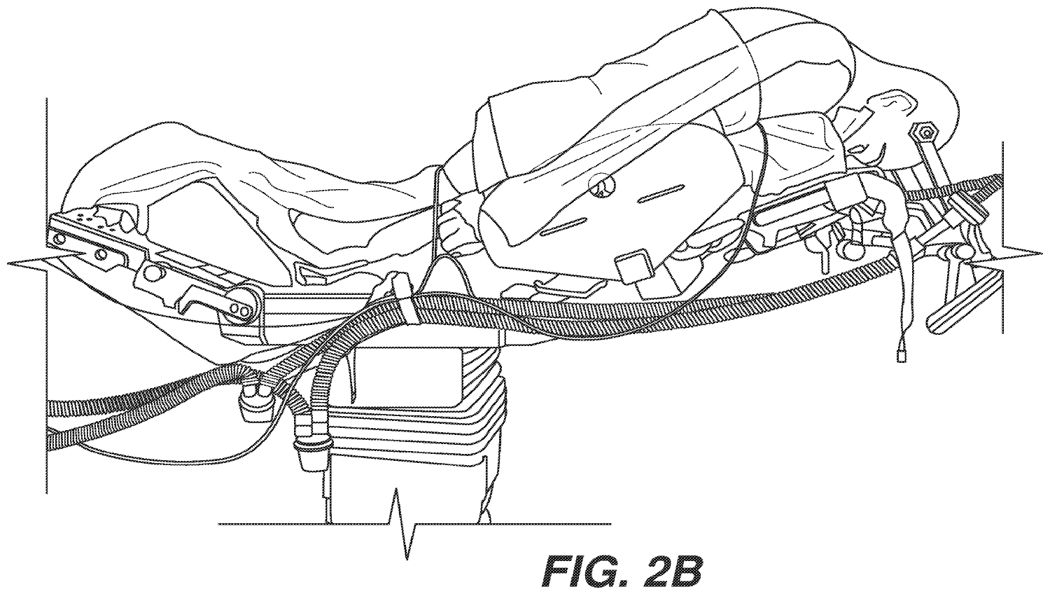

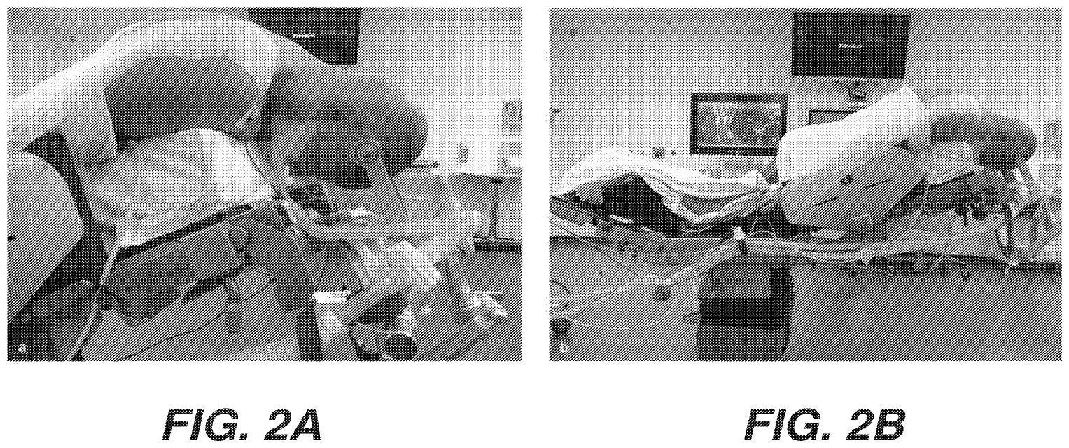

### DETAILED DESCRIPTION OF EXAMPLE EMBODIMENTS

The present disclosure relates generally to a registration tool or probe that includes one or more bends to enable localizer visibility irrespective of the localizer's orientation with respect to the patient. The bayonet tool is configured to have a curved shaft with the tool-tip pointing upwards. This design will enable the user to register a patient making sure the probe's marker is above the patient's plane. At certain angles, this might not be possible but due to the large marker shaft, even at steep angles, the marker will be present in the localizer's line of sight.

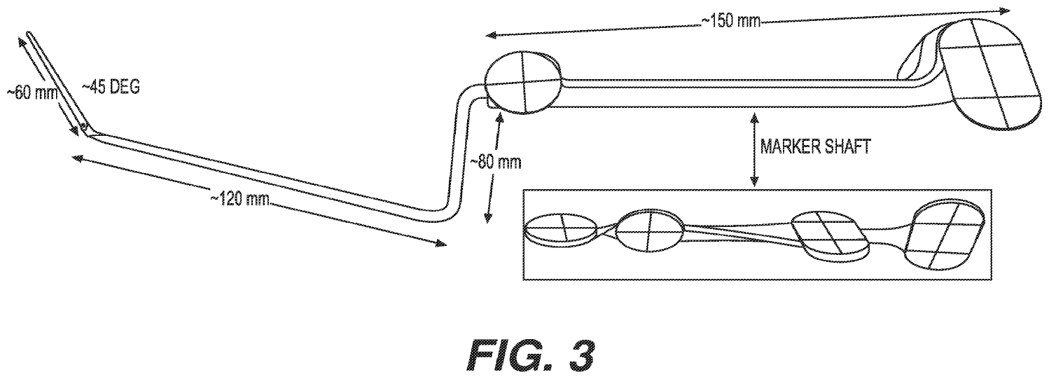

Since the registration probe marker will be within the line of sight in most cases, the user would not have to move the localizer position multiple times for registration. Based on the 99th percentile of human head size, the curved part shall have enough width (˜80 mm from center of shaft) to allow free movements around the patients head, as shown in FIG. 3. The curved/bent design will also enable the user to remain standing for most cases. However, depending on the case and the surgeons preference, they might bend down for better visibility.

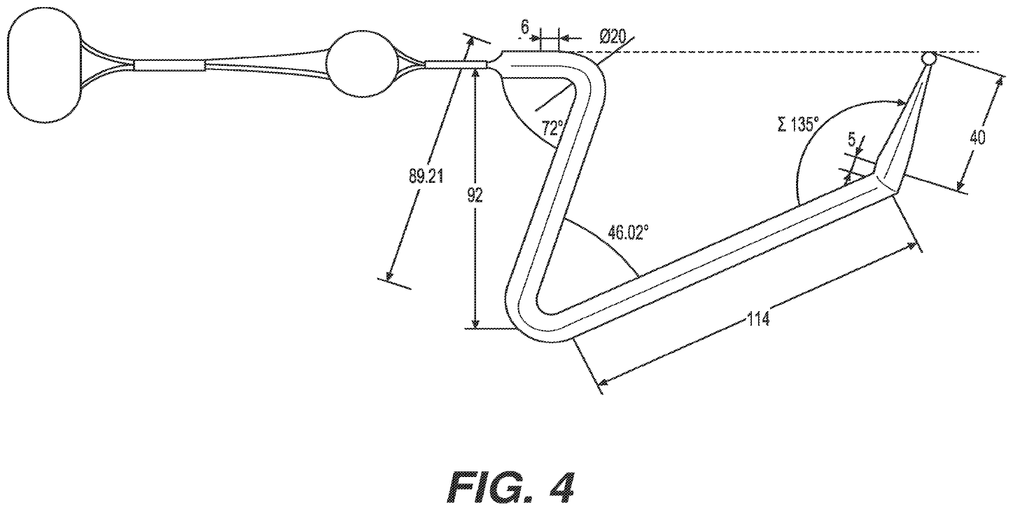

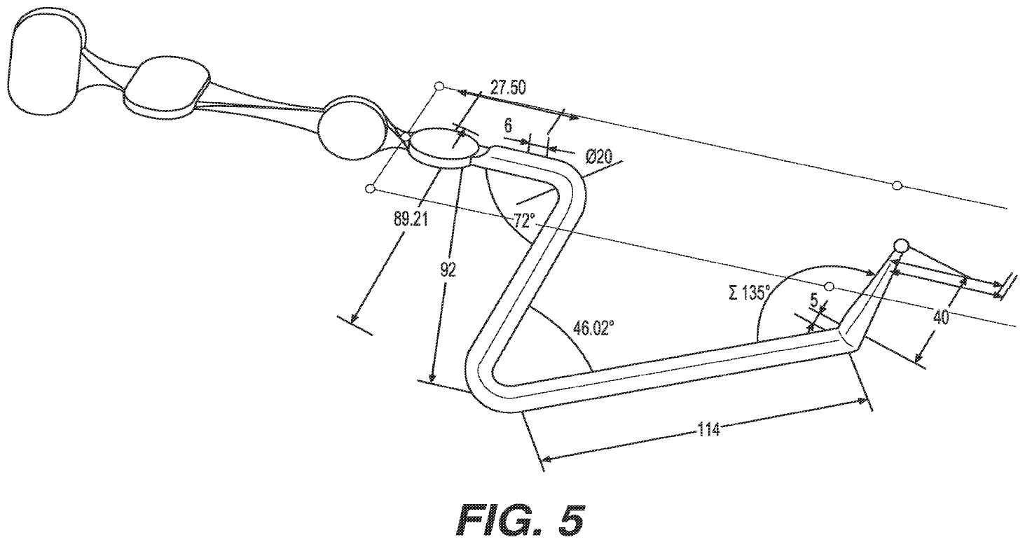

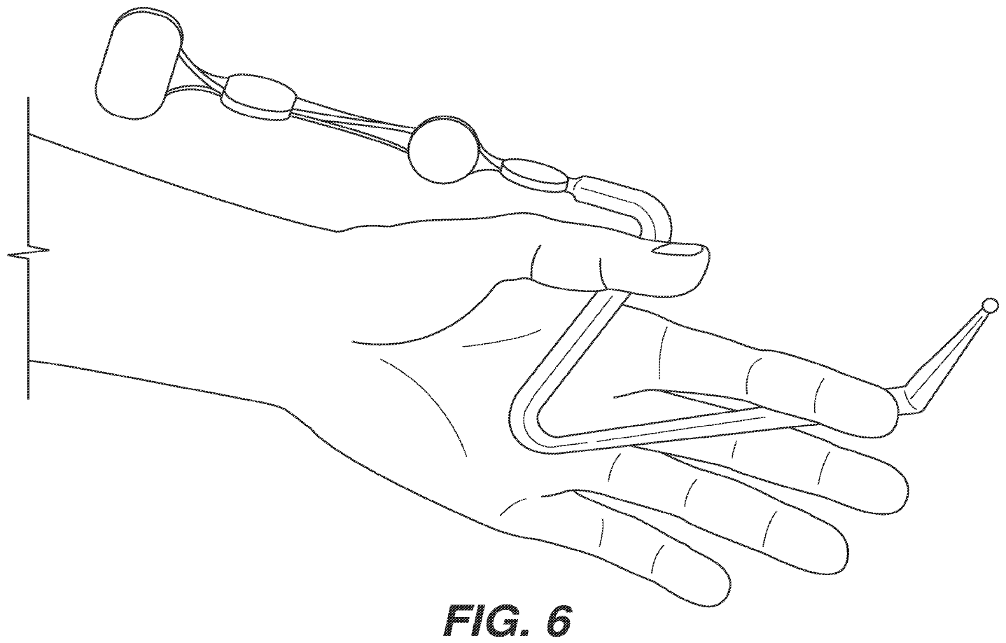

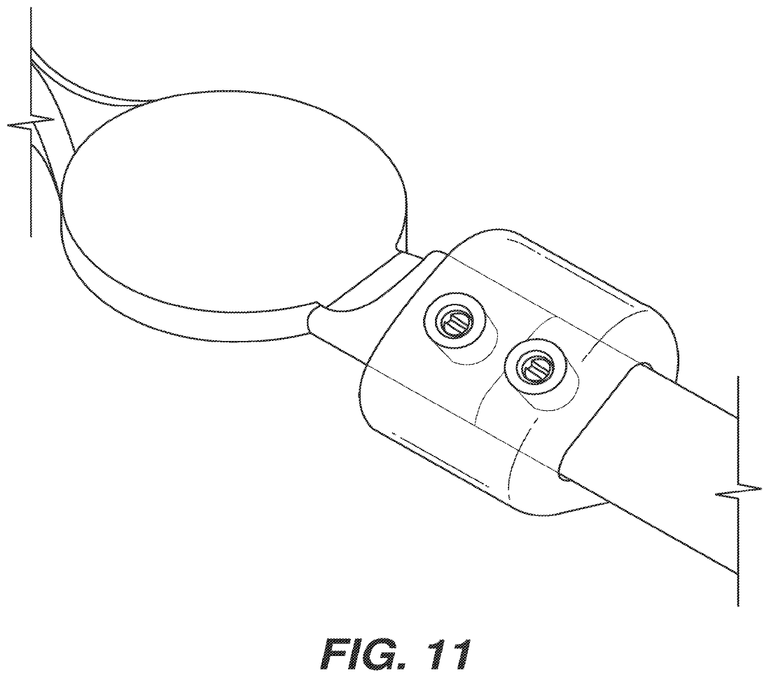

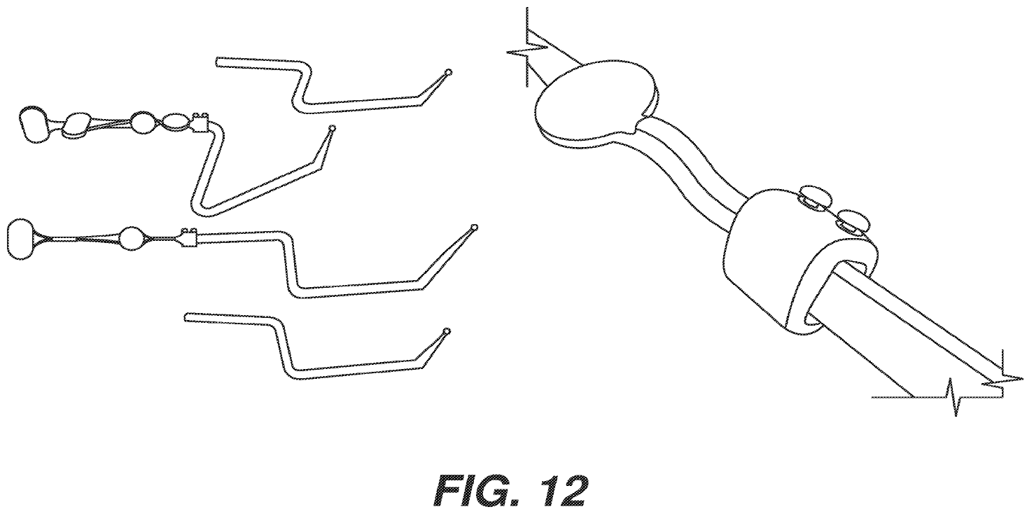

As shown in FIGS. 3 to 12 the registration tool includes a marker shaft 302 and a bent shaft 304. The tool may have a triangular shape. In some aspects, each side of the triangular shape may have a length of around 11.9 mm with rounded edges having a certain radius. In another embodiment (e.g., as shown in FIGS. 4 and 5), at least one side 402 of the triangular shape may be of lengths around 11.4 mm. The triangular shape or cross section provides better grip and stability when held by a user, as shown in FIG. 6. Any minor movements during registration adds error to the registration, where the triangular profile of the probe helps reduce or eliminate user unintended movement. A spherical ball 602 may be formed at a tip of the tool, having a radius around 3.9 mm.

In some embodiments, the shaft may be tapered 30 mm from a tip of the tool. In some embodiments, the registration tool may be made of AL 6061-T6. Additionally, in some embodiments, the tool may include one or more markers that are etched using sand blasting and/or acid etching 2-Step black anodize. The registration tool may be made from other materials/manufacturing methods in other embodiments.

The marker shaft may be 155 mm long and contain one or more marker disks (e.g., four marker disks). The markers may be oriented such that every other marker disc is oriented perpendicular to each other. This helps define four facets for the registration tool. The bent shaft may be bent in three segments. A first segment may have a length of 45 mm, a second segment has a length of 116.5 mm, and a third segment has a length of 42 mm. It should be appreciated that in other embodiments the lengths may change. An angle between the marker shaft and the first segment is about 45 to 90 degrees. An angle between the first segment shaft and the second segment is about 45 to 120 degrees. An angle between the second segment shaft and the third segment is about 45 to 120 degrees.

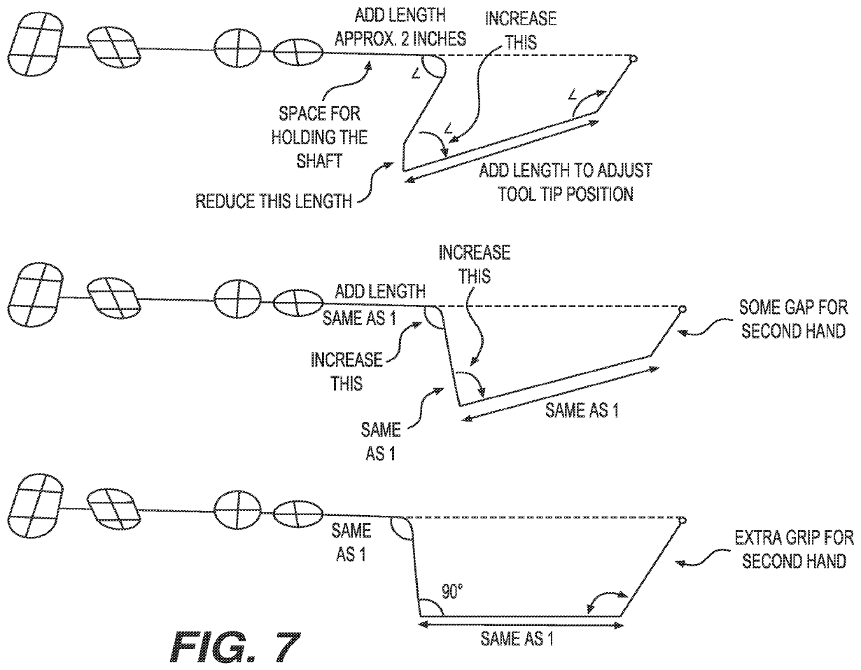

FIGS. 7 and 8 show variations among the segment lengths and angles. Despite the variations, the combination of segments and angles between the segments provides a registration tool that provide patient registration of prone patients for surgical navigation systems. For example, in registration probe 702, the length of the marker shaft may be extended approximately two inches, and the marker shaft may have a space for a user to hold. Furthermore the first segment of the bent shaft (e.g., the segment closest to the marker shaft) may have a reduced length. There may be an increased angle between the first and segment of the bent shaft, and the second segment may be lengthened to adjust for tool tip position.

As another example, in registration probe 704, the length of the marker shaft may also be extended to provide space for a user to hold. Furthermore, the angle between the marker shaft and the first segment of the bent shaft may be increased (e.g., so that it is above 90 degrees), the length of the first segment may be decreased, the angle between the first segment and the second segment of the bent shaft may be increased (e.g., so that is above 90 degrees), and the second segment may also be lengthened to adjust for tool tip position. In addition, the third segment of the bent shaft may have some grip (e.g., for a user to place their finger for extra support on the tool). Registration probe 706 may be similar to registration probe 704 but may also have an increased angle between the second and third segments of the bent shaft.

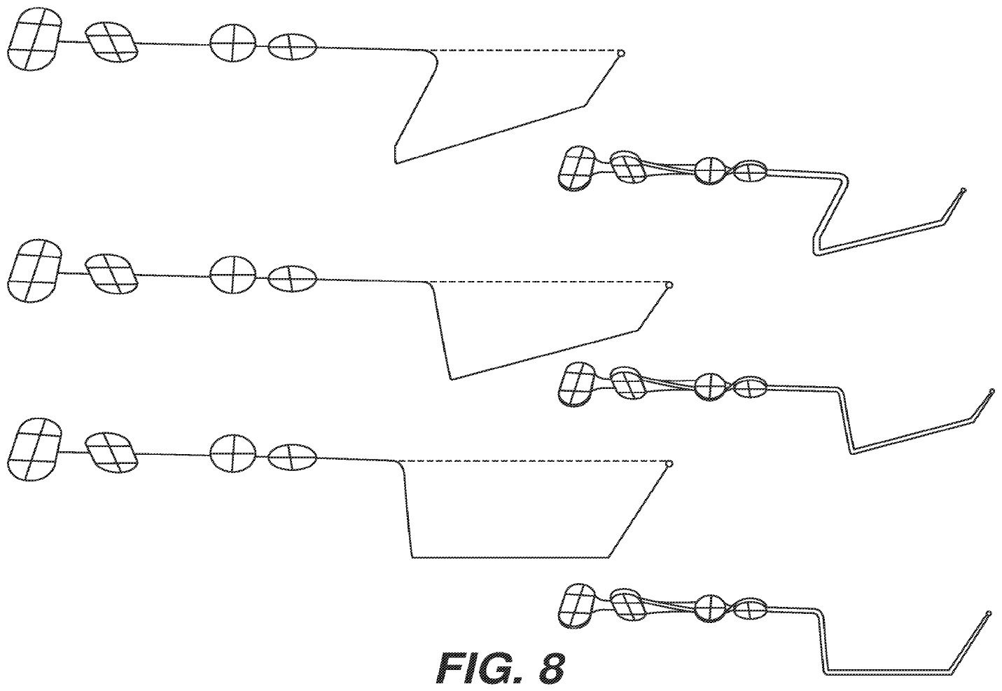

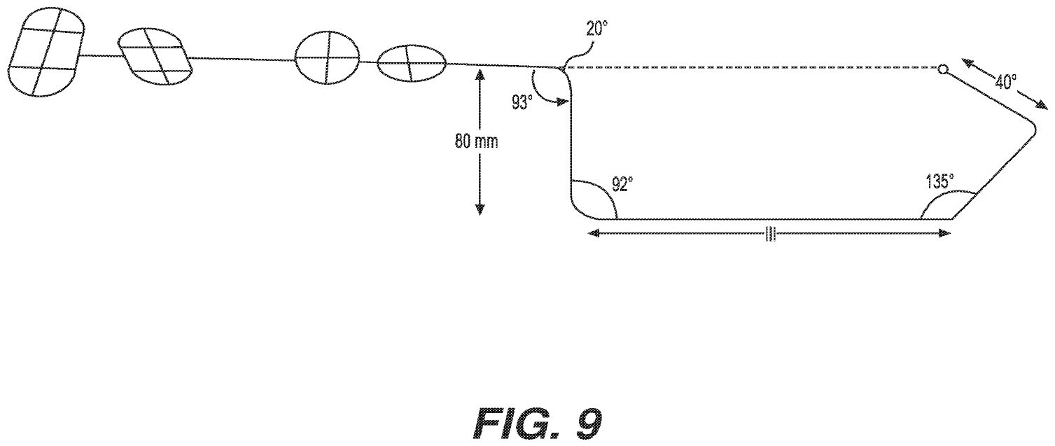

FIG. 9 shows another embodiment where a fourth segment 902 is added. The fourth segment 902 is angled inward to provide more freed to access a face of a patient while the localizer can still view the markers on the registration probe.

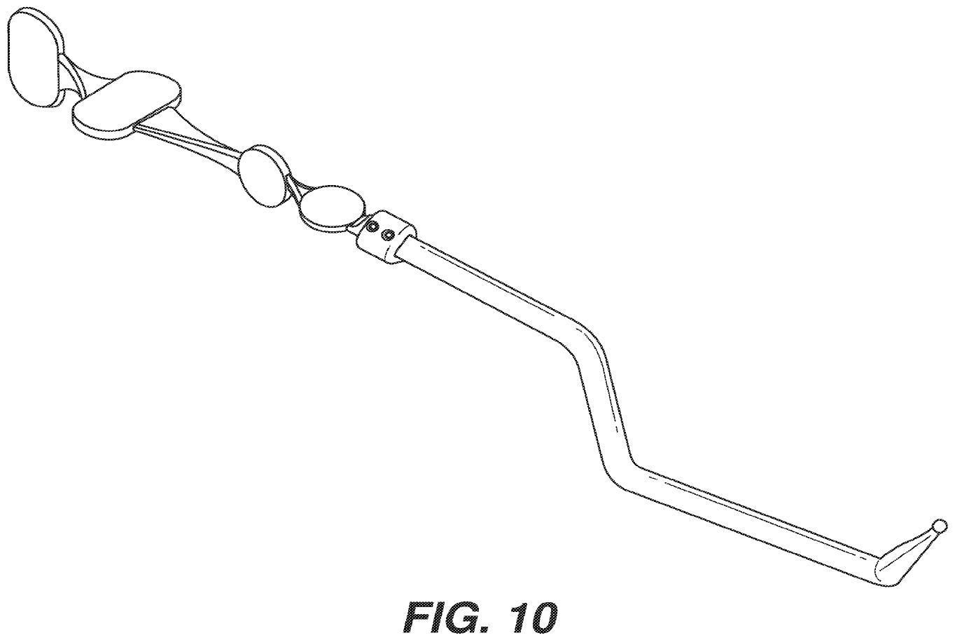

FIGS. 10 to 12 show an alternative design where the marker shaft is connected to the probe section with a coupling and set screws. In other embodiments, the marker shaft is connected to the probe section via a chemical fastener or other types of mechanical fasteners such as socket head cap screws.

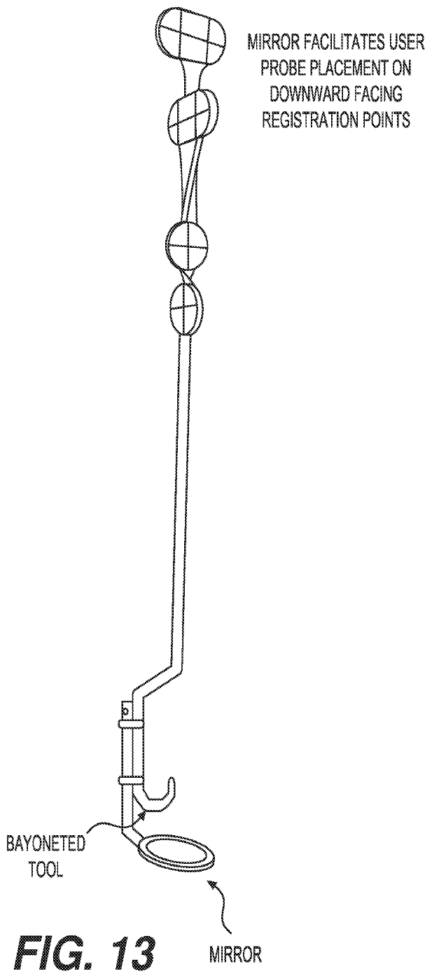

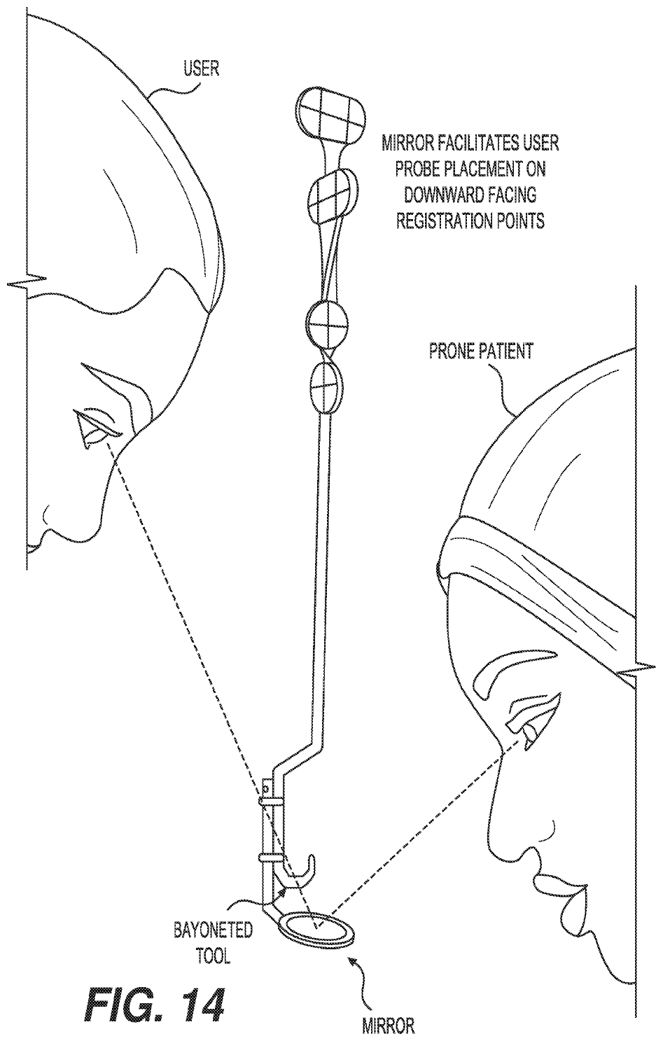

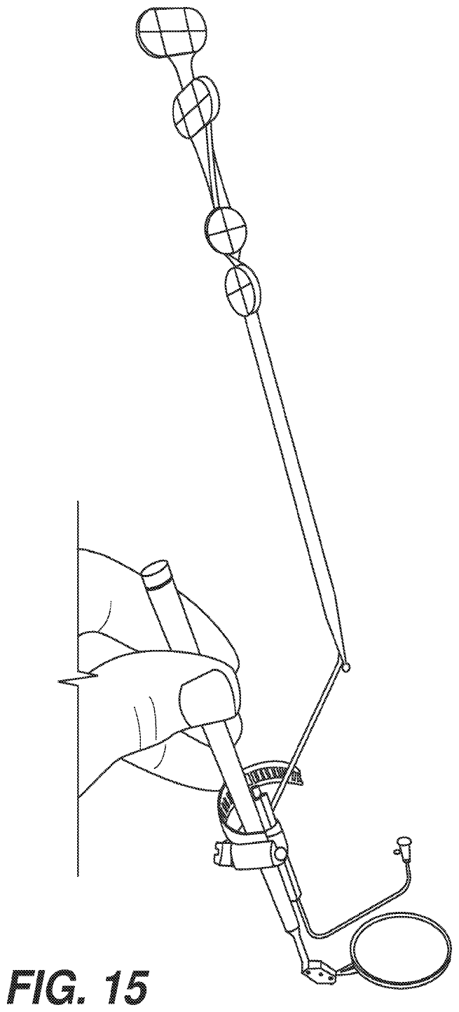

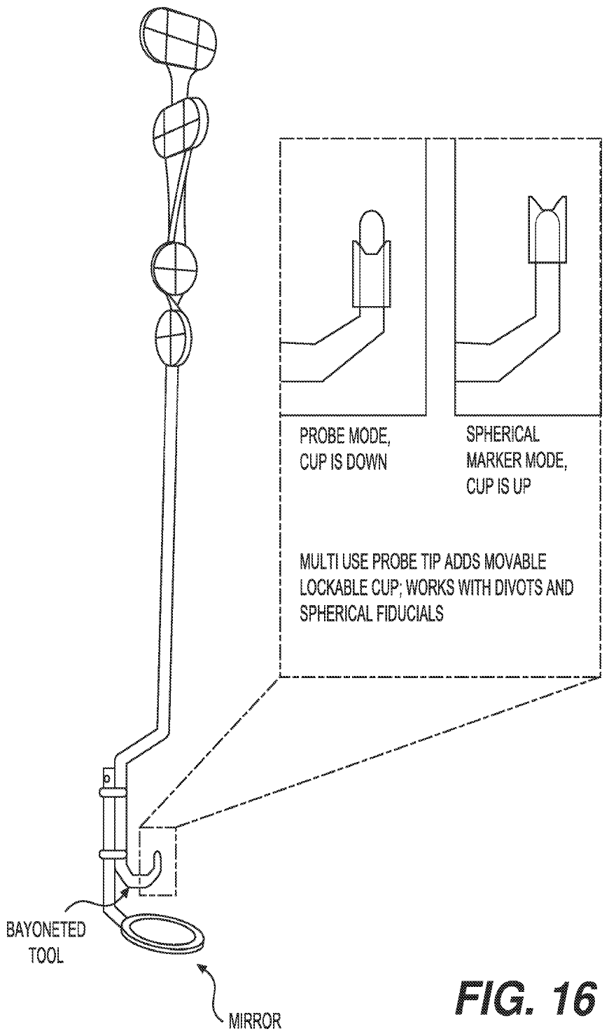

FIGS. 13 to 16 show that a mirror 1302 may be connected to an end or mid-section of the probe section. The mirror 1302 facilitates user probe placement on downward facing patient registration points. This enables a surgeon to handle the registration probe and align with a patient's facial features without having a direct visual line to the patient's face. The mirror 1302 is configured to reflect an end of the probe section back to a surgeon for placement against a patient's face. In some examples, the mirror 1302 is connected to the first and/or second section of the probe section 1304. In some embodiments, as shown in FIG. 16, the probe tip may serve multiple functions. For example, a movable and lockable cup, which may include a spherical and/or concaved marker, may be placed around the probe tip (e.g., by slipping the cup on the probe tip). In some aspects, the movable and lockable cup may be configured to attach to and/or roll along the probe tip, and work with divots and spherical fiducials. When the cup is slid downwards, exposing the probe tip, the probe tip may continue to be used as a probe (e.g., as shown in box 1602). However, if the cup remains along the edge of the probe tip, the cup may serve as a spherical and/or concave marker (e.g., as shown in box 1604).

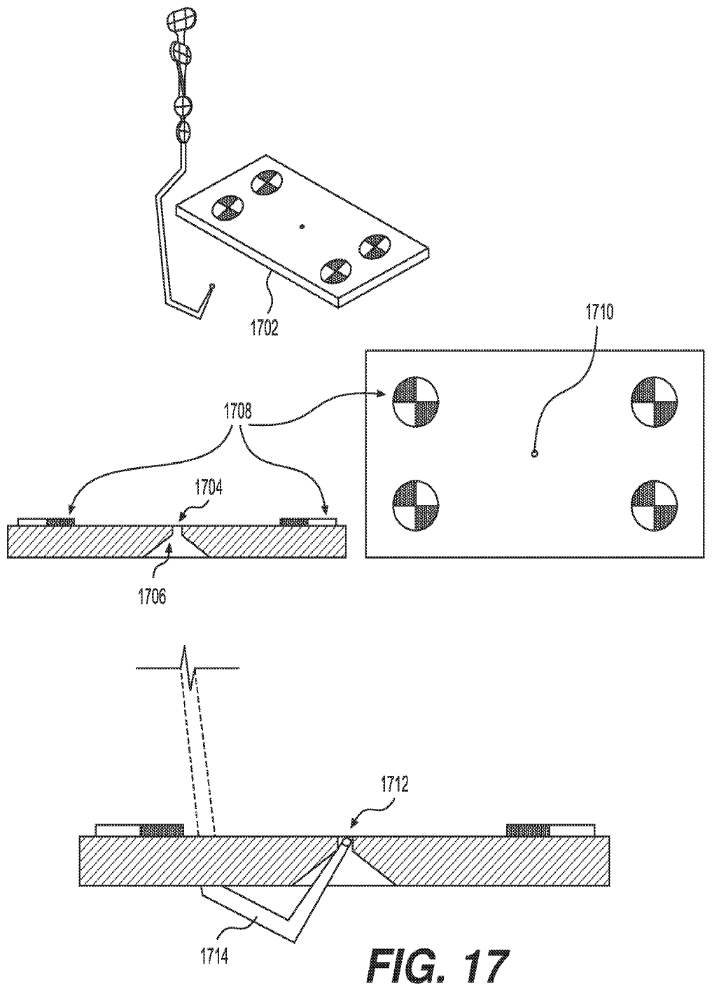

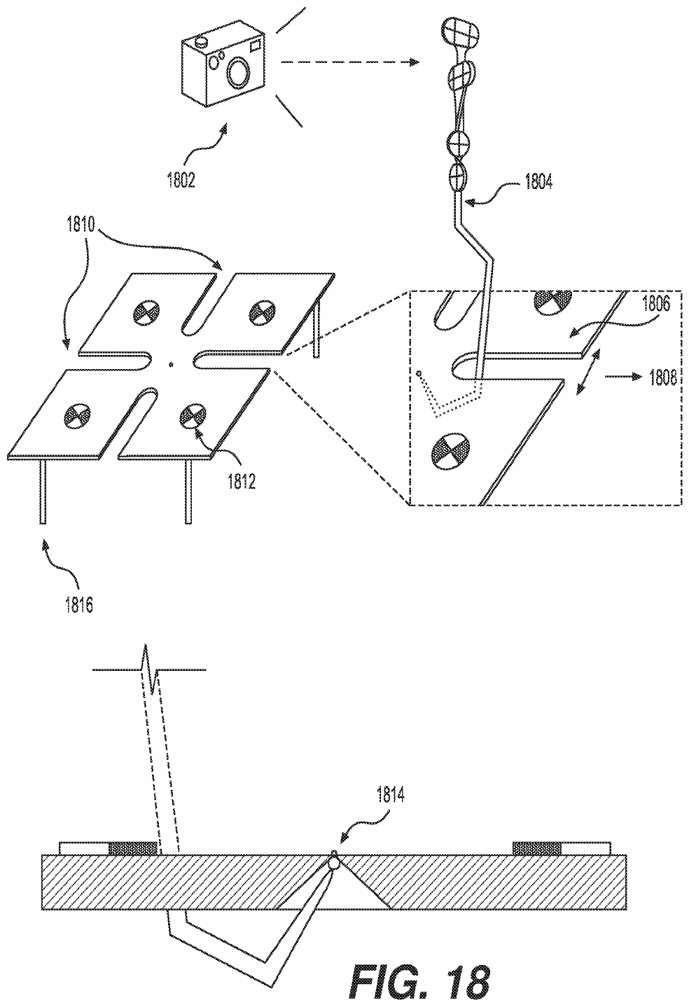

In some embodiments, as will be explained below in relation to FIGS. 17 and 18, a calibration plate may be used to secure the bayonet surgical registration tool in place. The calibration plate may be structured to allow the fiducials on the bayonet surgical registration tool to be easily detectable by the localizer.

FIG. 17 shows diagrams of an example calibration plate to secure the bayonet surgical registration tool, according to an example embodiment of the present disclosure. As shown in FIG. 17, the calibration plate may include a hole at the center 1702 to secure the tip of the bayonet registration probe in place. The hole may be drilled from the top 1704 and 1710 of the calibration place. However, the entry for the probe tip may be at the bottom 1706 of the calibration plate. The circumference of the hole on the top 1704 of the calibration plate may be smaller than the tip of the bayonet surgical registration probe in order to allow the registration probe to rest. However, the circumference of the hole at the bottom 1706 of the calibration plate may be larger than the bayonet surgical registration probe (e.g., thus forming a countersink) to allow the tip of the bayonet surgical registration probe to be inserted into the hole. Thus, the calibration plate may be structured to allow the entry of the tip of the surgical registration probe from the bottom side 1702. In some aspects, as shown in marker 1712, the tip of a bent surgical registration probe 1714 (e.g., bayonet surgical registration probe) may rest in the same location of the calibration plate as where a tip of a straight surgical registration probe would rest. Furthermore, the calibration plate may include one or more fiducials (e.g., four fiducials 1708 at each corner of the calibration plate) to serve as navigation targets for the localizer.

FIG. 18 shows diagrams of an example slitted calibration plate to more effectively secure the surgical registration tool, according to an example embodiment of the present disclosure. As shown in FIG. 18, the calibration plate allows the fiducials located on the shaft 1804 of the bayonet surgical registration probe to be easily detectable by the localizer 1802, for example, by causing the shaft 1804 of the bayonet surgical registration probe to remain perpendicular to the calibration plate. Furthermore, the calibration plate may have four slits 1810 and/or slots as shown. As shown in a close up for one of the slits (e.g., slit 1806), each slit may facilitate entry of the bayonet surgical registration probe. The slit may form a slot 1808 to allow a bent tool (e.g., the bayonet surgical registration probe) to go below the calibration plate. The slits 1810 may be interspersed between areas of the calibration plate showing fiducials 1812 to serve as navigation targets for the localizer 1802. Furthermore, the calibration plate may have a hole 1814 at the center for the tip of the bayonet surgical registration probe to rest.

In some embodiments, the slit (e.g., slit 1806) may allow the localizer 1802 to calibrate its detection of the registration probe multiple times (e.g., via fiducials on the shaft 1804 registration probe). For example, the slit 1806 may allow the registration probe to rotate and/or turn around within the slot 1808 in different orientations, thereby allowing the localizer 1802 to calibrate without having to move the localizer 1802 itself. This calibration can help improve the accuracy of detecting the surgical registration probe during surgery.

In some embodiments, the inner surface 1810 of the slits can have a layer 1806 comprising of rubber, silicone, an adhesive, and/or a friction-inducing substance to help hold the surgical registration probe in place and/or to reduce human-induced vibrations. In another embodiment, the calibration plate may have stilts 1816 to raise the height of the calibration plate. The stilts 1816 may allow for a part of the surgical registration probe to go below the calibration plate.

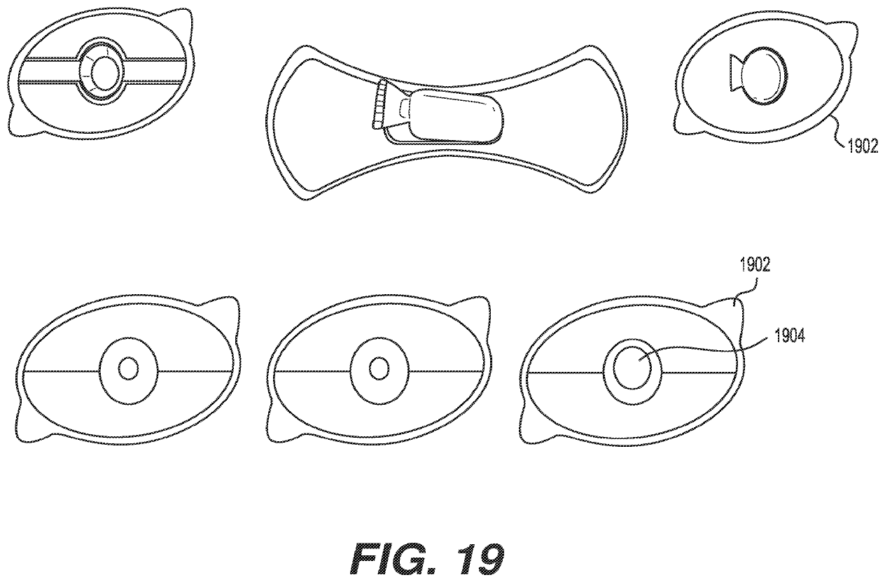

Just as the bayonet surgical registration probe can be secure via the calibration plate, other devices and/or apparatuses disclosed herein may be used to additionally or alternatively secure surgical registration probes. For example, as will be described in relation to FIG. 19, the bayonet surgical registration probe may be able to secure itself through physical complements added on to the patient (e.g., as a layer, adhesive, and/or surface).

FIG. 19 shows diagrams of example add-on patient markers configured to mate the bayonet surgical registration tool, according to an example embodiment of the present disclosure. As shown in FIG. 19, each add-on markers may have a mechanism for adhering to the skin or other surface of a patient. For example, an adhesive substance 1902 on the surface of the add-on patient marker that contacts the patient my facilitate adherence of the add-on patient marker to the patient. In some aspects, the add-on patient marker may allow a user to remove the add-on patient marker from the patient, e.g., via tabs 1904 on either side of the add-on patient marker. In some embodiments, the outward surface of the add-on patient marker may include fiducials and/or patient markers for the localizer to detect and use as a navigation target.

As shown in an example add-on patient marker of FIG. 19, an area 1906 of the add-on patient marker may be structured to hold and secure the tip of the surgical registration probe (e.g., the bayonet surgical registration probe). For example, the area 1906 may be outward facing from the patient, and may have a countersink hole drilled in to allow the tip of the surgical registration to probe and rest. The depth of the hole may be long enough to properly hold the surgical registration probe such that markers on the surgical registration probe remain stable enough to be detected by the localizer.

In various embodiments, the disclosed surgical registration probes (e.g., bayonet surgical registration probe) may be reusable after sterilization. For example, the sterilization may be performed using autoclave, ethylene oxide (EtO), or hydrogen peroxide (H₂O₂).

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims. To the extent that any of these aspects are mutually exclusive, it should be understood that such mutual exclusivity shall not limit in any way the combination of such aspects with any other aspect whether or not such aspect is explicitly recited. Any of these aspects may be claimed, without limitation, as a system, method, apparatus, device, medium, etc.

What is claimed is:

1. A surgical registration probe including:
a marker shaft with at least one marker or fiducial; and
a probe shaft having at least three sections that are bent at a defined angle with respect to each other.

2. The surgical registration probe of claim 1, wherein the marker shaft includes four markers or fiducials.

3. The surgical registration probe of claim 1, further comprising a mirror connected to at least one section of the probe shaft and positioned to reflect an end of the probe shaft.

4. The surgical registration probe of claim 1, wherein an end of the probe shaft includes a spherical ball for placement against a patient's skin.

5. The surgical registration probe of claim 1, wherein a first segment is connected to the probe shaft at an angle between 20 and 120 degrees, a second segment is connected to the first segment at an angle between 20 and 120 degrees, and a third segment is connected to the second segment at an angle between 20 and 120 degrees.

6. The surgical registration probe of claim 1, wherein the surgical registration probe is reusable after sterilization.

7. The surgical registration probe of claim 1, wherein the surgical registration probe is reusable after sterilization using one or more of:
autoclave,
ethylene oxide (EtO), or
hydrogen peroxide (H₂O₂).

8. A calibration plate for securing a surgical registration probe, the calibration plate comprising:
a rectangular plate comprising a top surface, a bottom surface, and a hole at the center of the rectangular plate having an opening through the top surface and an opening through the bottom surface;
wherein the top surface displays one or more fiducials;
wherein the one or more fiducials causes a localizer to track the calibration plate; and
wherein the hole is configured to secure a tip of the surgical registration probe via the opening through the bottom surface.

9. The calibration plate of claim 8,
wherein the surgical registration probe is a bayonet surgical registration probe comprising a marker shaft portion, a bent shaft portion, and the tip;
wherein the marker shaft portion comprises one or more additional fiducials causing the localizer to track the surgical registration probe; and
wherein the calibration plate is configured to secure the bayonet surgical registration probe by causing the marker shaft portion to be perpendicular to the rectangular plate, and causing the bent shaft portion to be underneath the bottom surface of the rectangular plate.

10. The calibration plate of claim 8,
wherein the opening through the top surface is smaller than the tip of the surgical registration probe; and
wherein the opening through the bottom surface is larger than the tip of the surgical registration probe.

11. The calibration plate of claim 8,
wherein the hole is configured as a countersink; and
wherein the opening through the bottom surface is larger than the opening through the top surface.

12. The calibration plate of claim 8, further comprising:
a plurality of stilts protruding from the bottom surface of the rectangular plate, wherein the plurality of stilts elevate the rectangular plate.

13. The calibration plate of claim 12, wherein the elevation of the rectangular plate is configured to allow a curved shaft of the surgical registration probe to be placed beneath the bottom surface of the rectangular plate.

14. The calibration plate of claim 8, further comprising:
a plurality of slits extending inwards from the sides of the rectangular plate, wherein the plurality of slits are configured to hold a shaft of the surgical registration probe.

15. An apparatus for improved localizer visibility to a surgical registration probe, the apparatus comprising:
a surgical registration probe;
a localizer configured to track the surgical registration probe; and
a surgical visualization system configured to display a surgical site responsive to the tracking;
wherein the surgical registration probe comprises:
a marker shaft with at least one marker or fiducial; wherein the marker or fiducial causes the localizer to detect the surgical registration probe; and
a probe shaft having at least three sections that are bent at a defined angle with respect to each other.

16. The apparatus of claim 15, wherein a first segment is connected to the probe shaft at an angle between 20 and 120 degrees, a second segment is connected to the first segment at an angle between 20 and 120 degrees, and a third segment is connected to the second segment at an angle between 20 and 120 degrees

17. The apparatus of claim 15, wherein an end of the probe shaft includes a spherical ball for placement against a patient's skin.

18. The apparatus of claim 15, further comprising:
a calibration plate for securing the surgical registration probe.

19. The apparatus of claim 17, wherein the calibration plate comprises:
a rectangular plate comprising a top surface, a bottom surface, and a hole at the center of the rectangular plate having an opening through the top surface and an opening through the bottom surface;
wherein the top surface displays one or more fiducials;
wherein the one or more fiducials causes a localizer to track the calibration plate; and
wherein the hole is configured to secure a tip of the surgical registration probe via the opening through the bottom surface.

20. The apparatus of claim 19, further comprising:
wherein the surgical registration probe is a bayonet surgical registration probe;
wherein the calibration is configured to secure the bayonet surgical registration probe by causing the marker shaft to be perpendicular to the rectangular plate, and causing the probe shaft to be underneath the bottom surface of the rectangular plate.
